(19) United States
(12) Patent Application Publication
(10) Pub. No.: US 2007/0093794 A1
(43) Pub. Date: Apr. 26, 2007

(54) Device, system and method for dual-path ophthalmic device

(76) Inventors: Qi Wang, West Jordan, UT (US); Dale Brown, West Pleasant View, UT (US)

Correspondence Address: PEARL COHEN ZEDEK LATZER, LLP, 1500 BROADWAY 12TH FLOOR, NEW YORK, NY, 10036, US

(21) Appl. No.: 11/399,623

(22) Filed: Apr. 7, 2006

Related U.S. Application Data
(60) Provisional application No. 60/726,221, filed on Oct. 14, 2005.

(51) Int. Cl. A61B 18/18

(52) U.S. Cl. 606/6

(57) ABSTRACT

This invention relates to a dual-path ophthalmic laser system designed for use by ophthalmologists for performing Selective Laser Trabeculoplasty (SLT) and Photodisruptive Procedure (PD) for the treatment of glaucoma and secondary cataract, respectively.

### BACKGROUND OF THE INVENTION

This invention relates to a dual-path ophthalmic laser system designed for use by ophthalmologists for performing Selective Laser Trabeculoplasty (SLT) and Photodisruptive Procedure (PD) for the treatment of glaucoma and secondary cataract, respectively. In particular, the invention relates to a dual-path ophthalmic laser system that may operate effectively in both the infrared region and at a wavelength in the visible region, for example, approximately in the green range of the spectrum, which may be suitable for glaucoma treatment.

In FIG. 1, the prior art of a dual-path ophthalmic laser system 100 is presented, as described in the US patent publication application (US 2004/0215175 A1). System 100 includes suitable optical devices that enables to selectively operate dual-path ophthalmic laser system 100 at two wavelengths, where at the first wavelength used for PD, the light path is enumerated by segment 173, and where at the a second wavelength used for SLT, the light path is enumerated by segments 174, 175, 176 and 178. According to the prior art arrangement, rotation of wave plate 120 may alter polarization direction of the light, whereas the polarization direction determines whether the light striking on polarizer 130 is transmitted to the PD output path 173 or reflected along path 174 to mirror 140 and to path 175, 176 and 178 to the SLT output.

### SUMMARY OF THE INVENTION

According to one aspect of the invention, an ophthalmic laser apparatus is disclosed which comprises a frequency multiplier capable of multiplying wavelength of a laser beam from a first wavelength to a second wavelength. The frequency multiplier may be displaceable into or out of a first light path. The apparatus may also comprises a separator capable of substantially transmitting the first wavelength and substantially reflecting the second wavelength to a second lightpath.

The apparatus of an embodiment of the present invention may also include a wave plate positioned in the first light path upstream of the separator for polarizing the laser beam having the first wavelength. The separator may be capable of substantially transmitting the first wavelength depending on the polarization direction of the laser beam.

The apparatus of an embodiment of the present invention may also include that the frequency multiplier generates the second wavelength from a portion of the first wavelength, wherein the amount of energy of the second wavelength generated depends upon the relation between the polarization direction of the first wavelength and the crystal structure of said frequency multiplier.

The method and apparatus of the present invention will be better understood by reference to the following detailed discussion of specific embodiments and the attached figures which illustrate and exemplify such embodiments.

### BRIEF DESCRIPTION OF THE DRAWINGS

The subject matter regarded as the invention is particularly pointed out and distinctly claimed in the concluding portion of the specification. The invention, however, both as to organization and method of operation, together with features and advantages thereof, may best be understood by reference to the following detailed description when read with the accompanied drawings in which:

FIG. 1 schematically depicts a prior art arrangement of a dual-path ophthalmic laser System;

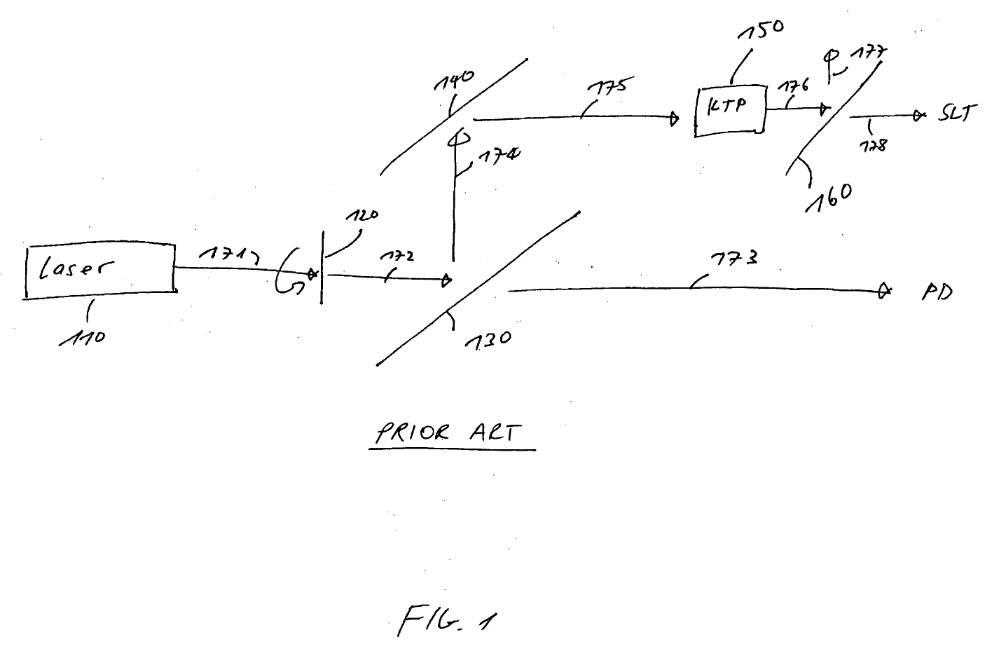

FIG. 2A schematically depicts a dual-path ophthalmic laser system in accordance with an embodiment of the present invention in a first configuration; and

FIG. 2B schematically depicts a dual-path ophthalmic laser system in accordance with an embodiment of the present invention in a second configuration.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, units and/or circuits have not been described in detail so as not to obscure the invention.

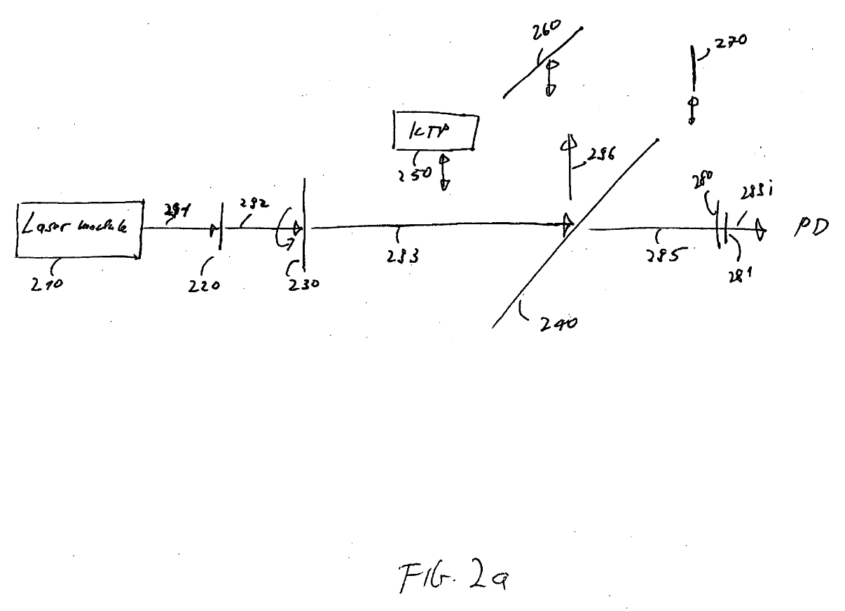

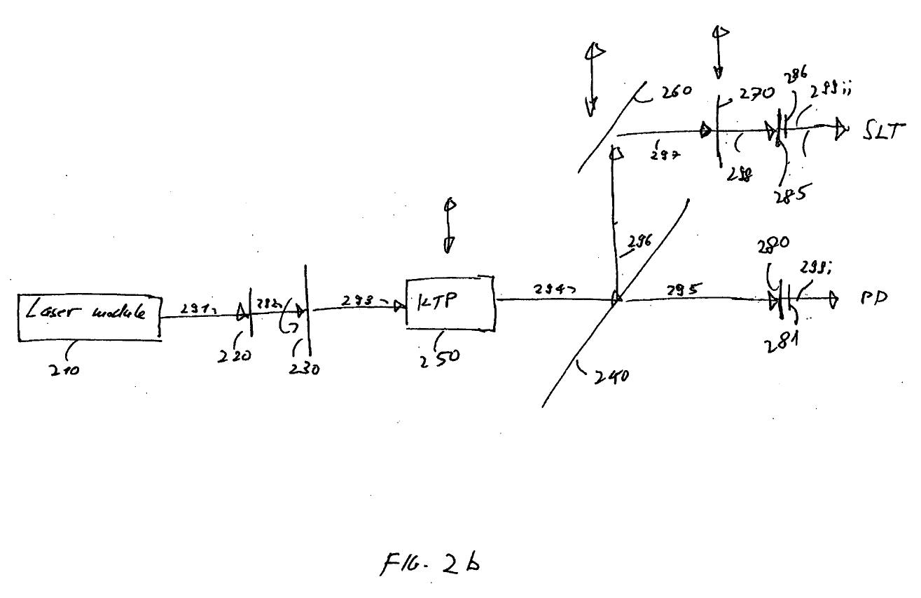

A dual-path ophthalmic laser system according to embodiments of the present invention may allow an operator to select a mode of treatment to be administered to a patient, by altering the configuration of the system, for example, by inserting a suitable optical element into the light path, for example, frequency doubler 250, as described in the embodiment below. Reference is made to FIG. 2A and FIG. 2B, which schematically depict two configurations of a dual-path ophthalmic laser system 200 in accordance with embodiments of the present invention. The system of the depicted embodiment may comprise laser module 210, visible light filter 220, wave plate 230, separator-polarizer 240, frequency doubler 250, green reflector 260 and IR filter 270 as depicted in FIG. 2A and FIG. 2B, in accordance with an embodiment of the invention. It may be noted that IR filter 270 may also be located in light path segments enumerated as 296 and 299ii.

The system according to embodiments of the present invention may be operated at two wavelengths, where at the first wavelength used for PD, the light path is enumerated by segment 295 and 299i and where at the second wavelength used for SLT, the light path is enumerated by segments 296, 297, 298 and 299ii.

Laser module 210 may be any suitable laser, including but not limited to a Q-switched Nd:YAG laser operating in the infrared spectrum, for example, at a wavelength of 1064 nm and a pulse width of, for example, less than 5 nsec. The light emitted from laser module 210 may be linearly polarized. Other laser modules, such as, for example Nd:YLF, Yb:YAG, may also be suitable as may be readily apparent to persons skilled in the art.

The light emitted from laser module 210 may be filtered by visible light filter 220. The light filtered of visible spectra at the filter output 292 may be incident to wave plate 230, which may be adjusted to change the polarization direction and/or rotation direction of light emitted therefrom. For example, introducing a half wave-plate, which may consist of a birefringent crystal, may result in phase retardation between o- and e-waves of the light, such that the polarization direction of a linearly polarized light may be altered.

The material of the polarizer may absorb one polarization pattern of the light more than another as the light propagates through the polarizer. A number of crystalline materials may be used as polarizer. In one embodiment of the invention, separator-polarizer 240 may have a multilayered thin film coating. Separator-polarizer 240 may reflect certain portions of the wavelength spectrum and transmit other portions. In some embodiments of the invention, the absorption effect of wavelengths by separator-polarizer 240 may be negligible. Thus, for example, in FIG. 2A, in which the frequency of the light striking separator-polarizer 240 is in the IR region, e.g., 1064 nm, the light may be partially transmitted to the PD light path 285. In the configuration of FIG. 2B, in which the frequency of the light may be doubled, for example, by placing KTP crystal 250 into the light path before the light strikes separator-polarizer 240, the frequency-doubled light, having frequency 532 nm may be reflected to SLT path 296.

Separator-polarizer 240 may transmit and/or reflect varying amounts of the incident light based on its polarization direction. In some embodiments of the invention, the reflectance at separator-polarizer 240 due to polarization direction may be mostly attributed to light, whose wavelength is in the IR region. Accordingly, by adjusting wave plate 230, the intensity of light in the IR region may be affected after being reflected off or transmitted by separator-polarizer 240. For example, the adjustment of wave plate 230 may lead to a P-polarization pattern of the light in the IR region striking on separator-polarizer 240 such that almost all of the light, e.g., more than 95%, for a wavelength of 1064 nm, may be transmitted through separator-polarizer 240 and therefore into the PD path 295. Conversely, if the adjustment of the wave plate 230 leads to an S-polarization pattern of the light, which may be in the IR region, almost the entire amount of the light, e.g., >98% for a wavelength of 1064 nm, may be reflected by separator-polarizer 240. Thus, the light may be reflected into the SLT path enumerated as 296, 297, 298 and 299ii.

In one configuration of an embodiment of the present invention, photodisruptive (PD) treatment may be desired. The optical setup of the dual-path ophthalmic laser system 200 for PD treatment may be as depicted in FIG. 2A. Wave plate 230 located after laser module 210 may be adjusted such that the polarization direction of the light at wave plate output 293 may result in the transmission of a desired amount of the light through separator-polarizer 240, as required by or suitable for the particular PD treatment. As a result, the energy of light output 295 after being transmitted by separator-polarizer 240 may have the energy required for the PD treatment, for example, 0.3-10 mJ in an 8-10 μm spot at 1064 nm wavelength.

In another configuration of an embodiment of the invention, it may be desired to generate light of a wavelength suitable for the SLT treatment. The optical setup for SLT treatment may be as indicated in FIG. 2B, which may be realized by inserting a frequency doubler 250, for example, a KTP crystal, between wave plate 230 and the separator-polarizer 240. The frequency doubler element may be placed into the light path manually, or automatically, for example by a motor activated by a button depressed by the operator of the device. In addition, a green reflector 260 and an IR filter 270 may be inserted after the light is reflected into path 296 and before the SLT output 298. It may be noted that in some embodiments of the invention, green mirror 260 and/or IR filter 270 may also be placed into the SLT light path manually or automatically.

Frequency doubler 250 may be inserted into dual-path ophthalmic laser system 200 between wave plate 230 and separator-polarizer 240 in order to modify the wavelength of the light at wave plate output 293 from the IR region to the visible green region, e.g., from 1064 nm to 532 nm, respectively.

Green reflector 260 may reflect light into the SLT optical path 296, which may lead to additional optics and to the patient's eye.

It will be noted that the light at wave plate output 293 may not all be doubled in frequency by frequency doubler 250. As a consequence, some portion of the light energy after the frequency doubler may be in the original, for example infrared, wavelength. Thus, the portion of the light in the IR region, together with the light in the green region, may be reflected by separator-polarizer 240 into path 296 towards the green reflector 260. However, due to the low reflectance of the green reflector 260 in the IR range, only a small portion of the light energy in the IR range may be reflected along SLT path 297. In order to fturther reduce the residual IR light energy in the light on SLT path, IR filter 270 may be inserted anywhere in the SLT path, for example, between separator-polarizer 240 and reflector 260, or together with reflector 260, or downstream from reflector 260, etc.

In some embodiments of the invention, light in the visible green region, which may be suitable for SLT treatment, may be almost fully reflected at separator-polarizer 240. Unlike the IR light, the reflectance of light in the visible green region at separator-polarizer 240 and as a consequence the intensity of the green light after being reflected at separator-polarizer 240 along the SLT path may not depend on the polarization direction of green light striking on separator-polarizer 240. The intensity of green light may rather depend on the polarization direction of the IR light incident to frequency doubler 240. The polarization direction of the IR light and as a consequence, the second harmonic generation efficiency at frequency doubler 250 may be changed, which determines the green light intensity at frequency doubler output 294. Therefore, the adjustment of wave plate 230 determines the green light intensity in the SLT path. The light at path 298 may have an energy density at which SLT treatment is desired, for example, e.g., 0.01-5 J/cm² at 532 nm wavelength.

In some embodiments of the invention, shutters 280 and 285 may be located at separator-polarizer output 295 and/or IR filter output 298, respectively. When the path suitable for PD treatment is in use, the path suitable for SLT treatment may be closed by shutter 285 and vice versa. To avoid scattering of the light at the shutters 280 and/or 285, absorbers 281 and/or 286, respectively, may be used on the shutters, whereas a shutter and an absorber may form a shutter blade.

Other wavelengths may be suitable for other ophthalmic applications, in which case the frequency doubler 250 may triple or quadruple the wavelength of the light emitted from laser module 210. In some applications, it may be desirable to use a tunable frequency doubler, such as an optical parametric oscillator. Other optical elements, for example, lenses, beam shapers, attenuators and the like may be used in conjunction with embodiments of the present invention.

What is claimed is:

1. An ophthalmic laser apparatus, comprising:
a frequency multiplier capable of multiplying wavelength of a laser beam from a first wavelength to a second wavelength, said frequency multiplier being displaceable into or out of a first light path; and
a separator capable of substantially transmitting said first wavelength and substantially reflecting said second wavelength to a second lightpath.

2. The apparatus of claim 1, further comprising a wave plate positioned in the first light path upstream of said separator for polarizing said laser beam having said first wavelength, wherein said separator is capable of substantially transmitting said first wavelength depending on said polarization direction of said laser beam.

3. The apparatus of claim 1, wherein said first wavelength has a polarization direction, wherein said frequency multiplier generates said second wavelength from a portion of said first wavelength, and wherein the amount of energy of said second wavelength generated depends upon the relation between the polarization direction of said first wavelength and the crystal structure of said frequency multiplier.

4. The apparatus of claim 1, wherein said frequency multiplier is a frequency doubler.

5. The apparatus of claim 1, wherein said frequency multiplier is a Potassium Titanyl Phosphate crystal.

6. The apparatus of claim 1, further comprising:
a filter filtering wavelengths of the infra-red spectrum of said laser beam.

7. The apparatus of claim 1, further comprising:
a visible light filter to filter light placed before said frequency multiplier to reduce visible light.

8. A method to obtain two laser beams each at a different wavelength, the method comprising:
multiplying a wavelength of a laser beam from a first wavelength to a second wavelength by introducing a frequency multiplier into a first light path;
substantially transmitting said first wavelength through a separator; and
substantially reflecting said second wavelength by said separator to a second light-path.

9. The method of claim 8, wherein separator and laser beam further comprise:
adjusting a wave plate positioned in the light path upstream of said separator to change polarization direction of said laser beam.

10. The method of claim 8, wherein said reflecting and transmitting depend on the polarization direction of said laser beam.

11. The method of claim 8, further comprising:
generating a second wavelength from a portion of said first wavelength by a frequency doubler, said portion depending upon the relation between the polarization direction of said first wavelength and the crystal structure of said frequency doubler.

12. The method of claim 8, further comprising:
filtering infra-red spectrum out of said laser beam.

13. The method of claim 8, further comprising:
filtering green spectrum out of said laser beam.

14. An ophthalmic laser beam apparatus, comprising:
a wave plate positioned in a path of a laser beam to change the polarization direction of said laser beam;
a separator positioned in said path for directing the laser beam depending upon its wavelength; and
a frequency multiplier positioned between said wave plate and said separator, wherein said frequency multiplier is adapted for moving into or out of said light path.

15. The ophthalmic laser beam apparatus of claim 14, wherein said laser beam has a first wavelength, and wherein said frequency multiplier is further adapted to generate a second wavelength from a portion of said first wavelength, said portion depending upon the relation between the polarization direction of said first wavelength and the crystal structure of said frequency multiplier.
